(19) United States
(12) Patent Application Publication
(10) Pub. No.: US 2025/0195387 A1
(43) Pub. Date: Jun. 19, 2025

(54) HAIR STRAIGHTENING COMPOSITIONS AND METHODS FOR STRAIGHTENING HAIR

(71) Applicant: L'OREAL, Paris (FR)

(72) Inventors: Cho-Cho KHINE, Scotch Plains, NJ (US); Ronak RUGHANI, Edison, NJ (US)

(73) Assignee: L'OREAL, Paris (FR)

(21) Appl. No.: 18/539,865

(22) Filed: Dec. 14, 2023

(51) Int. Cl. A61K 8/73; A45D 7/06; A61K 8/362; A61Q 5/04

(52) U.S. Cl. CPC A61K 8/738; A45D 7/06; A61K 8/362; A61K 8/737; A61Q 5/04

(57) ABSTRACT

The present disclosure is drawn to a hair straightening composition and methods for straightening hair. The hair straightening composition includes: (a) citric acid, a salt thereof, or a combination thereof; (b) one or more cyclodextrins, derivatives thereof, or a combination thereof; (c) one or more water soluble solvents; (d) one or more cationic guar derivatives; and (e) water. After application of the hair straightening composition to the hair, the hair is physically straightened and dried, optionally with heat. Hair treated with the hair straightening composition exhibits improved fiber alignment, frizz control, and smoothness.

### FIELD OF THE DISCLOSURE

The present disclosure is drawn to a hair straightening composition and methods for straightening hair. Hair treated with the hair straightening composition exhibits improved fiber alignment, frizz control, and smoothness.

### BACKGROUND

Many consumers use cosmetic and care compositions to enhance the appearance of hair, e.g., by changing the color, style, or shape of the hair and/or by imparting various cosmetic properties to hair, such as shine and conditioning. Hair can become dry or damaged for various reasons, e.g., weather exposure, poor nutrition, mechanical treatments (e.g., brushing hair), styling treatments using chemicals, dying, heat, nutrition, etc. Even cleansing products can remove hair's natural oils causing dryness, which can lead to a dull appearance, split ends, and frizz.

Chemical treatments for hair include bleaching and coloring treatments to change the color the hair. Chemical treatments also include processes to permanently change the shape and structure of the hair, for example by perming, waving, relaxing or straightening the hair. These chemical treatments change the look of hair by changing its physical structure, which inevitably causes a certain degree of damage to the hair. Environmental factors, such as salt water, sunlight, and heat, are also known to damage hair. Damaged hair is characterized by unnatural changes to the protein structure of the individual hair strands or shafts.

The popularity and usage of oils for hair treatments has increased due to their effectiveness and simplicity. Commonly used oils include olive oil, mineral oil, avocado oil, apricot kernel oil, rice bran oil, and coconut oil. However, these treatments can leave the hair feeling greasy. In addition, the effects are not usually seen after more than several hours (e.g., 8 hours) of treatment and several treatments are usually required, making it time consuming and labor intensive.

Damage to hair results in split ends, dryness, hair that is easily broken, and hair that becomes “frizzy” and unmanageable. Because the visible portion of hair is dead, it has no ability to regenerate itself. There are numerous over the counter and salon treatments that purport to repair damaged hair. These include conditioners, hot oil treatments, hydrolyzed proteins, vitamin formulations, and exotic fruit, leaf, or root extracts. These treatments, however, provide only limited improvement to the hair. Therefore, hair treatment technologies that can straighten, relax, or style the hair without chemically damaging the hair are desired.

There is still a need for providing improved hair straightening, smoothing, and manageability, for example, improved hair alignment, reduced unwanted volume (especially reduced frizz), and increased shine.

Further, the formulation of environmentally friendly cosmetic products, which are designed and developed considering environmental issues, is becoming a major goal in an effort to meet global challenges. It is therefore essential to propose more sustainable compositions, preparation processes and ingredients to address these environmental concerns. In this context, it is important to develop new cosmetic formulations with a better carbon footprint, particularly by promoting the use of renewable raw materials and/or cosmetic compositions with a good index of naturalness and/or materials of natural origin and, more particularly, materials of plant origin while reducing the use of compounds of petrochemical origin.

### SUMMARY OF THE DISCLOSURE

The instant disclosure is drawn to hair straightening compositions and methods for straightening and smoothing hair. Hair treated with the compositions exhibits improved fiber alignment, reduced frizz, and smoothness. Further, the hair is soft, shiny, conditioned, and has a healthy appearance. The compositions include a combination of citric acid, or salt thereof, and a cyclodextrin, or derivative thereof. The citric acid, or salt thereof, and the cyclodextrin, or derivative thereof, are separately combined with each other and the cyclodextrin or derivative is dissolved in the citric acid. Subsequently, the combination is incorporated into the hair straightening composition.

The hair straightening compositions typically include:
(a) citric acid, a salt thereof, or a combination thereof;
(b) one or more cyclodextrins, derivatives thereof, or a combination thereof;
(c) one or more water soluble solvents;
(d) one or more cationic guar derivatives; and
(e) water.

Citric acid and its salts provide a variety of benefits to hair. For example, it acts as an antioxidant, removes build-up and debris from the hair, and improves blood circulation in the scalp, which in turn nourishes hair follicles and promotes growth. Its acidic pH is also useful for balancing the pH of the scalp, as many hair care products make are alkaline. The citric acid in the hair straightening compositions also interacts with cyclodextrin in a unique manner and improves film formation on the hair, which is further potentiated with heat.

Cyclodextrins are a family of cyclic oligosaccharides, comprised of a macrocyclic ring of glucose subunits joined by α-1,4 glycosidic bonds. Cyclodextrins are produced from starch by enzymatic conversion. Typical cyclodextrins contain a number of glucose monomers ranging from six to eight units in a ring, creating a cone shape, for example, α (alpha)-cyclodextrin gas 6 glucose subunits, β (beta)-cyclodextrin has 7 glucose subunits, and γ (gamma)-cyclodextrin has 8 glucose subunits. Nonlimiting examples of cyclodextrins for use in the compositions of the present disclosure include α-cyclodextrin, β-cyclodextrin, γ-cyclodextrin, methyl-α-cyclodextrin, methyl-β-cyclodextrin, methyl-γ-cyclodextrin, and mixtures thereof.

The weight ratio of the citric acid, salts thereof, or combination of (a) to the cyclodextrin or derivatives of (b) is typically from about 4:1 to about 1:1 ((a):(b)) and/or the mole ratio of the citric acid, salts thereof, or combination of (a) to the cyclodextrin or derivatives of (b) is typically from about 20:1 to about 5:1 ((a):(b)).

The citric acid, salts thereof, or combination of (a) and the cyclodextrin, derivatives thereof, or combination thereof of (b) are typically combined with one another before being combined with the other ingredient of the hair straightening composition. For example, the cyclodextrin and/or derivatives thereof (cyclodextrin component) are preferably dissolved or solubilized in the citric acid, salt thereof, or combination thereof (citric acid component) to form a solubilized combination of citric acid and cyclodextrin. The combination of cyclodextrin component and citric acid component can be heated to facilitate or hasten the dissolution of the cyclodextrin component in the citric acid component. When the cyclodextrin component is individually dissolved in the citric acid component prior to combination with other ingredients of the hair straightening compositions, the cyclodextrin/citric acid component provides unique properties to the hair straightening composition that differ from the properties that result from combining the cyclodextrin component and the citric acid component with the other ingredients of the composition without first dissolving the cyclodextrin component in the citric acid component.

Nonlimiting examples of water soluble solvents include C₂-C₆ mono-alcohols (e.g., ethanol, isopropyl alcohol, etc.), polyols (polyhydric alcohols), glycerin, glycols (e.g., ethylene glycol, propylene glycol, butylene glycol, pentylene glycol, caprylyl glycol, etc.), and mixtures thereof. Glycerin, also known as glycerol or glycerine, is a simple triol (C₃H₈O₃). It is a colorless, odorless, and viscous liquid that acts as a natural humectant by attracting and moisture from its surroundings.

Cationic guar derivatives include cationically modified guar and hydroxypropyl guar and alkyl guar, in particular, cationic hydroxylpropyl guar, as well as salts thereof. A typical cationic functional group in these cation guar derivatives is trimethylamino (2-hydroxyl) propyl, with a counter ion such as a halide (e.g., chloride, fluoride, bromide, iodide), sulfate, or methylsulfate. Nonlimiting exemplary cationic guar derivatives include cationically modified alkylated carboxylated polymers such as methyl and ethyl carboxymethyl guar and cationic hydroxypropyl guar, in particular, cationic hydroxypropyl guar.

The hair straightening compositions are aqueous compositions containing significant amounts water. In addition to water, the compositions optionally include one or more water soluble solvents other than glycerine. Nonlimiting examples of water-soluble solvents include C₂-C₆ mono-alcohols (e.g., ethanol, isopropyl alcohol, etc.), polyols (polyhydric alcohols), glycols (e.g., ethylene glycol, propylene glycol, butylene glycol, pentylene glycol, caprylyl glycol, etc.), and mixtures thereof. In various embodiments, at least one of the one or more water soluble solvents is glycerin, a glycol (e.g., ethylene glycol, propylene glycol, butylene glycol, pentylene glycol, caprylyl glycol, etc.) or mixtures thereof.

The pH of the hair straightening compositions is typically acidic, for example, from about 3 to about 6.5, preferably from about 3 to about 6, more preferably from about 3 to about 5.

The hair straightening composition may optionally include one or more miscellaneous ingredients, for example, one or more preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or mixtures thereof.

Methods for straightening hair employing the hair straightening compositions include:
(i) applying a hair straightening composition of the instant disclosure to hair;
(ii) physically manipulating the hair upon which the hair straightening composition is applied with a straightening action;
(iii) without removing the straightening composition from the hair, drying the hair; and
(iv) optionally, heating the hair to a temperature of about 140° C. to about 220° C., wherein the heating of the hair may optionally be carried out after a pause time.

The hair straightening composition is applied to the hair and the hair can be combed, brushed, or pulled, for example, with fingers and hands, to straighten the wet or damp hair. A combing or brushing action helps ensure the hair straightening composition is uniformly spread throughout the hair while simultaneously straightening the hair. During or after carrying out the straightening action, the hair is dried. The hair can dry naturally under ambient conditions, for example, at a temperature of about 25° C. The hair can optionally be treated with a thermal treatment (a heat treatment) to dry and/or to shape the hair. A heat treatment is not required but in various embodiments, a heat treatment is preferred.

The heat treatment may be preceded by a pause time. A “pause time” is a period of time between application of the hair shaping composition to the hair and treating the hair with a heat treatment. For example, the hair shaping composition may be applied to the hair, after which a pause time is employed before treating the hair with a heat treatment. The pause time begins after the hair shaping composition has been applied to the hair before the hair becomes dry. After the pause time, the hair is treated with a heat treatment, for example, with a blow dryer or a hot iron, such as a curling iron or a flat iron, at a temperature of about 140° C. to about 220° C. The heat treatment can be used to aid in drying the hair and contribute to shaping of the hair or the heat treatment can be applied to the hair after the hair is dried, e.g., after a pause time. The pause time can vary but is typically from about 1 minute to about 2 hours. In a preferred embodiment, the hair is treated with a hair shaping composition and after a pause time of about 1 minute to about 1 hour, the hair is treated with a heat treatment, wherein the heat treatment is preferably treatment with a hot iron, such as a curling iron or a flat iron. The hair may dry naturally during the pause time or can be dried with a blow dryer.

### BRIEF DESCRIPTION OF THE DRAWINGS

Implementation of the present technology is described, by way of example only, with reference to the attached figures, wherein:

FIG. 1(a) compares hair swatches treated with an inventive composition, comparative compositions, and a control, after naturally drying overnight under ambient conditions;

FIG. 1(b) compares hair swatches treated with an inventive composition, comparative compositions, and a control, after naturally drying overnight under ambient conditions, and after 1 cycle of post treatment cleansing and drying (1×);

FIG. 1(c) hair swatches treated with an inventive composition, comparative compositions, and a control, after naturally drying overnight under ambient conditions, and after 2 cycles of post treatment cleansing and drying (2×);

FIG. 1(d) compares hair swatches treated with an inventive composition, comparative compositions, and a control, after naturally drying overnight under ambient conditions, and after 3 cycles of post treatment cleansing and drying (3×);

FIG. 2(a) compares hair swatches treated with an inventive composition, comparative compositions, and a control, followed by treatment with a flat iron, immediately after being treatment with the flat iron;

FIG. 2(b) compares hair swatches treated with an inventive composition, comparative compositions, and a control, followed by treatment with a flat iron, and after 1 cycle of post treatment cleansing and drying (1×);

FIG. 2(c) compares hair swatches treated with an inventive composition, comparative compositions, and a control, followed by treatment with a flat iron, and after 5 cycles of post treatment cleansing and drying (5×);

FIG. 2(d) compares hair swatches treated with an inventive composition, comparative compositions, and a control, followed by treatment with a flat iron, and after 6 cycles of post treatment cleansing and drying (6×);

FIG. 3(a) shows a curl pattern 7 hair swatch that was untreated, hair swatches treated with an inventive composition without heat, hair swatches treated with an inventive composition with heat, and a control hair swatch, after being subjected to 80% RH for 4 hours;

FIG. 3(b) shows a curl pattern 7 hair swatch that is untreated, hair swatches treated with an inventive composition without heat, hair swatches treated with an inventive composition with heat, and a control hair swatch, after being subjected to 80% RH for 4 hours, and after 1 post-treatment cleansing and drying cycle (1×); and

FIG. 3(c) shows a curl pattern 7 hair swatch that is untreated, hair swatches treated with an inventive composition without heat, hair swatches treated with an inventive composition with heat, and a control hair swatch, after being subjected to 80% RH for 4 hours, and after 3 post-treatment cleansing and drying cycles (3×).

It should be understood that the various aspects are not limited to the arrangements and instrumentality shown in the drawings.

### DETAILED DESCRIPTION OF THE DISCLOSURE

The instant disclosure is drawn to hair straightening compositions and methods for straightening hair. Hair treated according to the disclosure exhibits improved fiber alignment, reduced frizz, and smoothness. The hair straightening effects can be enhanced with heat, for example, with a flat iron, but heating is not necessary. Regardless of whether heat is employed, the straightening effects are long-lasting and durable, even under high humidity conditions. The hair straightening composition typically includes:
(a) about 1 to about 8 wt. % of citric acid, a salt thereof, or a combination thereof;
(b) about 0.5 to about 5 wt. % of one or more cyclodextrins, derivatives thereof, or a combination thereof;
   wherein a combined total amount of (a) and (b) is about 2 to about 12 wt. %;
(c) about 0.1 to about 5 wt. % of one or more water soluble solvents;
(d) about 0.1 to about 8 wt. % of one or more cationic guar derivatives; and
(e) about 70 to about 96 wt. % of water;
   wherein all percentages by weight are based on a total weight of wherein all percentages by weight are based on a total weight of the composition.

### (a) Citric Acid and Salts Thereof

The total amount of citric acid, salts thereof, or combination thereof will vary. Nonetheless, in various embodiments, the hair straightening composition includes about 1 to about 8 wt. % of citric acid, salts thereof, or a combination thereof, based on a total weight of the composition. In further embodiments, the hair straightening composition includes about 1 to about 6 wt. %, about 1 to about 4 wt. %, about 1 to about 3 wt. %, about 2 to about 8 wt. %, about 2 to about 6 wt. % about 2 to about 5 wt. %, about 2 to about 4 wt. %, about 2 to about 3 wt. %, about 2.5 to about 8 wt. %, about 2.5 to about 6 wt. %, about 2.5 to about 5 wt. %, about 2.5 to about 4 wt. %, about 2.5 to about 3 wt. %, or about 1, about 1.5, about 2, about 2.5, about 3, about 3.5, about 4, about 4.5, or about 5 wt. % of citric acid, salts thereof, or a combination thereof, based on a total weight of the composition.

### (b) Cyclodextrin and Derivatives

The hair straightening compositions include one or more cyclodextrins or derivatives thereof. As used herein, the term “cyclodextrins” includes slats of carboxylic acid, whether or not expressly stated. Cyclodextrins are a family of cyclic oligosaccharides consisting of a macrocyclic ring of glucose subunits joined by α-1,4 glycosidic bonds.

The cyclodextrins that can be used include those of the formula:

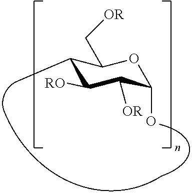

wherein:
R is chosen from H, CH₃, or a hydroxypropyl group, and
n ranges from 6-8.

For example, in embodiments where R═H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R═CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodexctrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

In one embodiment, the hair straightening compositions according to the present disclosure includes β-cyclodextrin in an amount ranging from about 0.1% to about 10%, such as from 0.2% to about 8%, from about 0.3% to about 7%, from about 0.4% to about 6%, from about 1% to about 10%, from about 1% to about 8%, from about 1% to about 5%, from about 1% to about 3% by weight, relative to the total weight of the hair straightening composition.

The total amount of cyclodextrin in the hair straightening compositions will vary. Nonetheless, in various embodiments, the hair straightening composition includes about 0.5 to about 5 wt. % of cyclodextrin, based on a total weight of the composition. In further embodiments, the hair straightening composition includes 0.5 to about 4 wt. %, about 0.5 to about 3 wt. %, about 0.5 to about 2 wt. %, about 1 to about 5 wt. %, about 1 to about 4 wt. %, about 1 to about 3 wt. %, about 1 to about 2 wt. %, about 1.5 to about 5 wt. %, about 1.5 to about 4 wt. %, about 1.5 to about 3 wt. %, about 1.5 to about 2 wt. %, about 0.5 wt. %, about 1 wt. %, about 1.25 wt. %, about 1.5 wt. %, about 2 wt. %, about 2.5 wt. %, about 3 wt. %, about 3.5 wt. %, about 4 wt. %, about 4.5 wt. %, or about 5 wt. %, based on a total weight of the composition.

### Combination of Citric Acid/Salts and Cyclodextrin

The total combined amount of the citric acid, salts thereof, or combination of (a) and the cyclodextrin or derivatives of (b) will vary. Nonetheless, in various embodiments the total combined amount of the citric acid, salts thereof, or combination of (a) and the cyclodextrin and derivatives of (b) is about 1 to about 12 wt. %, based on a total weight of the hair straightening composition. In further embodiments, the total combined amount of the citric acid, salts thereof, or combination of (a) and the cyclodextrin and derivatives of (b) is about 1 to about 10 wt. %, about 1 to about 8 wt. %, about 1 to about 6 wt. %, about 1 to about 5 wt. %, about 2 to about 12 wt. %, about 2 to about 10 wt. %, about 2 to about 8 wt. %, about 2 to about 6 wt. %, about 2 to about 5 wt. %, about 3 to about 12 wt. %, about 3 to about 10 wt. %, about 3 to about 8 wt. %, about 3 to about 6 wt. %, about 3 to about 5 wt. %, or about 1 wt. %, 2 wt. %, 3 wt. %, 4 wt. %, 5 wt. %, 6 wt. %, 7 wt. %, or 8 wt. %, based on a total weight of the hair straightening composition.

The weight ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a weight ratio of about of about 8:1 to about 1:2 ((a):(b)). In further embodiments, the citric acid, salts thereof, or combination of (a) and the cyclodextrin or derivatives of (b) are in a weight ratio of 6:1 to about 1:2, about 5:2 to about 1:2, about 4:1 to about 1:2, about 3:1 to about 1:2, about 2:1 to about 1:2, about 8:1 to about 1:1, about 6:1 to about 1:1, about 5:1 to about 1:1, about 4:1 to about 1:1, about 3:1 to about 1:1, about 2:1 to about 1:1, about 1.2:1, about 1.3:1, about 1.4:1, about 1.5:1, about 1.6:1, or about 1.8:1 ((a):(b)).

The mole ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a mole ratio of about 20:1 to about 3:1. In further embodiments, the citric acid, salts thereof, or combination of (a) and the cyclodextrin or derivatives of (b) are in a mole ratio of about 18:1 to about 3:1, about 15:1 to about 3:1, about 20:1 to about 5:1, about 18:1 to about 5:1, about 15:1 to about 5:1, about 20:1 to about 8:1, about 18:1 to about 8:1, about 15:1 to about 8:1, about 20:1 to about 10:1, about 18:1 to about 10:1, about 15:1 to about 10:1, about 14:1, about 13:1, about 12:1, or about 11:1.

In various embodiments, the citric acid, salts thereof, or combination of (a) and the cyclodextrin of (b) are combined with one another before being added into the hair straightening compositions of the instant disclosure. For example, the cyclodextrin is preferably solubilized in the citric acid to form a solubilized combination of citric acid and cyclodextrin. The combination can be heated to facilitate or hasten the dissolution of the cyclodextrin. The solubility of cyclodextrin in water is not always ideal. Therefore, combining the cyclodextrin with the citric acid and dissolving the cyclodextrin in citric acid before adding combining the combination with other components of the hair straightening composition can be beneficial.

### (c) Water Soluble Solvents

The term “water soluble solvent” is interchangeable with the terms “water soluble organic solvent” and “water-miscible solvent” and means a compound that is liquid at 25° C. and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, organic solvents selected from glycerin, alcohols (for example C₁₋₈, or C₁₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from alcohols (for example C₂₋₆ or C₂₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, and propane diol.

Further non-limiting examples of water soluble organic solvents include alkanediols (polyhydric alcohols) such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In a preferred embodiment, the multiphase cleansing composition includes one or more glycols selected from propylene glycol, butylene glycol, pentylene glycol, hexylene glycol, caprylyl glycol, dipropylene glycol, and mixtures thereof.

In a preferred embodiment, the multiphase cleansing composition include glycerin.

The amount of water soluble solvents in the hair straightening compositions will vary. Nonetheless, the total amount of one or more water soluble solvents may be from about 0.1 to about 20 wt. %, based on the total weight of the hair straightening composition. In further embodiments, the hair straightening composition include from about 0.1 to about 15 wt. %, about 0.1 to about 10 wt. %, about 0.1 to about 5 wt. %, about 1 to about 20 wt. %, about 1 to about 15 wt. %, about 1 to about 10 wt. %, about 1 to about 5 wt. %, based on the total weight of the hair straightening composition.

In further embodiments, the hair straightening composition includes from about 0.1 to about 5 wt. % of one or more water soluble solvents, based on the total weight of the hair straightening composition. In further embodiments, the hair straightening composition includes from about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.5 to about 5 wt. %, about 0.5 to about 4 wt. %, about 0.5 to about 3 wt. %, about 0.5 to about 2 wt. %, about 0.8 to about 5 wt. %, about 0.8 to about 4 wt. %, about 0.5 to about 3 wt. %, about 0.8 to about 2 wt. % of one or more water soluble solvents, based on the total weight of the hair straightening composition.

In even further embodiments, the hair straightening composition includes from about 0.1 to about 5 wt. % of glycerin, based on the total weight of the hair straightening composition. In additional embodiments, the hair straightening composition includes from about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.5 to about 5 wt. %, about 0.5 to about 4 wt. %, about 0.5 to about 3 wt. %, about 0.5 to about 2 wt. %, about 0.8 to about 5 wt. %, about 0.8 to about 4 wt. %, about 0.5 to about 3 wt. %, about 0.8 to about 2 wt. % of glycerin, based on the total weight of the hair straightening composition.

### (d) Cationic Guar Derivatives

Cationic guar derivatives include cationically substituted galactomannan (guar) gum derivatives. The guar molecule itself is a straight chain mannan branched at regular intervals with single membered galactose units on alternative mannose units. The mannose units are linked to each other by means of β (1-4) glycosidic linkages. The galactose branching arises by way of an α (1-6) linkage. Cationic derivatives of the guar gums are obtained by reaction between the hydroxyl groups of the polygalactomannan and reactive quaternary ammonium compounds. The degree of substitution of the cationic groups onto the guar structure must be sufficient to provide a desired cationic charge density. Suitable quaternary ammonium compounds for use in forming the cationic guar polymers include those conforming to the general Formula (XII):

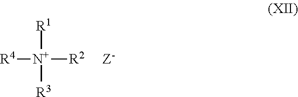

wherein where R1, R2 and R3 are methyl or ethyl groups;
R4 is either an epoxyalkyl group of the general Formula (XIII):

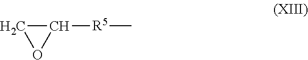

or R4 is a halohydrin group of the general Formula (XIV):

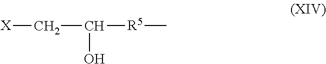

wherein R5 is a C1 to C3 alkylene; X is chlorine or bromine, and Z is an anion such as Cl—, Br—, I— or HSO4-.

Cationic guar polymers (cationic derivatives of guar gum) formed from the reagents described above are represented by the general Formula (XV):

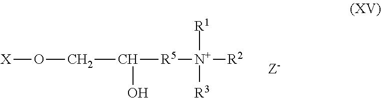

wherein R is guar gum. Preferably, the cationic guar polymer is guar hydroxypropyltrimethylammonium chloride, which can be more specifically represented by the general Formula (XVI):

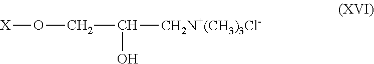

Suitable cationic guar derivatives have cationic charge densities of at least about 1.25 meq/gm, preferably at least about 1.5 meq/gm, more preferably at least about 1.7 meq/gm, even more preferably at least about 1.8 meq/gm, still more preferably at least about 2.0 meq/gm, and even more preferably at least about 2.3 meq/gm, but also preferably less than about 7 meq/gm, more preferably less than about 5 meq/gm, still more preferably less than about 4.5 meq/gm. The “cationic charge density” of a cationic guar derivative, as that term is used herein, refers to the ratio of the number of positive charges on a monomeric unit of which the polymer is comprised to the molecular weight of said monomeric unit. The cationic charge density multiplied by the polymer molecular weight determines the number of positively charged sites on a given polymer chain.

Any anionic counterions may be use in association with the cationic guars so long as the cationic guars remain soluble in water, in the hair straightening composition, or in a coacervate phase of the hair straightening composition, and so long as the counterions are physically and chemically compatible with the essential components of the hair straightening composition or do not otherwise unduly impair product performance, stability or aesthetics. Nonlimiting examples of such counterions include: halides (e.g., chloride, fluoride, bromide, iodide), sulfate, methylsulfate, and mixtures thereof.

Nonlimiting examples of cationic guar derivatives include cationic hydroxyethyl guar, cationic hydroxypropyl guar, cationic hydroxybutyl guar, cationic carboxymethyl guar, cationic carboxylpropyl guar, cationic carboxybutyl guar, carboxymethylhydroxypropyl guar, guar hydroxypropyltrimonium chloride, and hydroxypropyl guar hydroxypropyltrimonium chloride.

The amount of the cationic guar derivative in the hair straightening compositions will vary. Nonetheless, in various embodiments, the hair straightening composition includes from about 0.1 to about 8 wt. % of the cationic guar derivative, based on the total weight of the hair straightening composition. In further embodiments, the hair straightening composition includes from about 0.1 to about 5 wt. %, about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.5 to about 5 wt. %, about 0.5 to about 4 wt. %, about 0.5 to about 3 wt. %, about 0.5 to about 2 wt. %, about 0.8 to about 5 wt. %, about 0.8 to about 4 wt. %, about 0.5 to about 3 wt. %, about 0.8 to about 2 wt. % of the cationic guar derivative, based on the total weight of the hair straightening composition.

### (e) Water

The total amount of water in the hair straightening compositions will vary. Nonetheless, in various embodiments, the hair straightening compositions include about 70 to about 96 wt. % of water, based on the total weight of the compositions. In further embodiments, the hair straightening composition includes about 75 to about 96 wt. %, about 80 to about 96 wt. %, about 85 to about 96 wt. %, about 90 to about 96 wt. %, about 70 to about 95 wt. %, about 75 to about 95 wt. %, about 80 to about 95 wt. %, about 85 to about 95 wt. %, about 90 to about 95 wt. %, based on the total weight of the hair straightening composition.

### Miscellaneous Ingredients

The compositions optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the compositions, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

pH

The pH of the composition will vary. Nonetheless, the compositions typically have a pH of less than 7 (an acidic pH). For example, the pH can be from about 2.5 to about 6.5, about 2.5 to about 6, about 2.5 to about 5.5, about 2.5 to about 5, about 2.5 to about 4.5, about 2.5 to about 4, about 3 to about 6.5, about 3 to about 6, about 3 to about 5.5, about 3 to about 5, about 3 to about 4.5, or about 3 to about 4.

### Methods

As explained throughout the instant disclosure, the hair straightening compositions are particularly useful in methods for straightening hair, preferably human hair, in particular human hair of the head. Treatment with the compositions improves fiber alignment, reduces frizz, and impart smoothness to the hair. The methods typically comprise applying a hair straightening composition of the instant disclosure to the hair. The hair is preferably wet or damp, but the composition can also be applied to dry hair. Application of the compositions to the hair will moisten the hair because the compositions are aqueous (contain a large portion of water).

The hair straightening composition is spread throughout the hair, preferably spread substantially uniformly throughout the hair, as a leave-in product, i.e., the hair straightening composition is not typically rinsed from the hair prior to straightening and/or drying the hair. The hair upon which the hair straightening composition is applied can be physically straightened or physically manipulated with a straightening action. For example, a comb or brush can be used to incorporate the hair straightening composition throughout the hair while simultaneously straightening the hair, i.e., pulling the hair in a manner that straightens the hair. Similarly, the hair can be physically manipulated with one's hands to spread the hair straightening composition throughout the hair and the hands can also be used to pull or lay the hair flat to straighten the hair.

While the hair is being brushed or physically manipulated with a straightening action, the hair can dry naturally under ambient conditions. Alternatively, a blow dryer or hair dryer can be used to expedite drying of the hair. For example, the hair can be combed or brushed while being blow dried to simultaneously straighten and dry the hair. The blow dryer can provide heat to the hair and expedite the straightening and drying process. In various embodiments, the hair is subjected to a thermal treatment (heat treatment), i.e., the hair is heated to a temperature of about 120° C. to about 250° C., preferably 140° C. to about 250° C., more preferably about 150° C. to about 200° C. In various embodiments, after the hair is dry or is mostly dry, the hair can be treated with a thermal treatment (treated with heat). For example, the hair can be treated with a hot iron, in particular, a flat iron. Typically, the hot iron is passed over the hair at least once, at least twice, at least three times, or more. The hot iron is preferably at a temperature of about 120° C. to about 250° C., preferably 140° C. to about 250° C., more preferably about 150° C. to about 200° C.

In various embodiments, the method includes a pause time between application of the hair straightening composition to the hair and a thermal treatment. The pause time begins immediately after application of the hair straightening composition to the hair, while the hair still wet. The hair may dry naturally during the pause time, or the hair can be subjected to a drying process to expedite drying of the hair, for example, the hair can be dried with a blow dryer, provided the blow dryer does not heat the hair according to a subsequent thermal treatment (heat treatment). Otherwise, the blow drying is considered a thermal treatment according to the instant disclosure. After a pause time, a thermal treatment (heat treatment) is applied to the hair. For example, the thermal treatment preferably heats the hair to a temperature of about 120° C. to about 250° C., preferably 140° C. to about 250° C., more preferably about 150° C. to about 200° C. A blow dryer can provide the heat treatment provided the blow dryer heats the hair to the expressed temperatures. In a preferred embodiment, the hot iron is used for the heat treatment, of example, a curling iron or a flat iron, wherein the hot iron heats the hair to a temperature of about 120° C. to about 250° C., preferably 140° C. to about 250° C., more preferably about 150° C. to about 200° C.

When a pause is employed between application of the hair straightening composition and a thermal treatment, the pause time will vary but is typically from about 1 minutes to about 2 hours. In further embodiments, the pause time is from about 1 minute to about 1 hour, about 1 minute to about 30 minutes, about 1 minute to about 20 minutes, about 2 minutes to about 2 hours, about 2 minutes to about 1 hour, about 2 minutes to about 30 minutes, about 2 minutes to about 20 minutes, about 5 minutes to about 2 hours, about 5 minutes to about 1 hour, about 5 minutes to about 30 minutes, about 5 minutes to about 20 minutes, about 15 minutes to about 2 hours, about 15 minutes to about 1 hour, or about 15 minutes to about 30 minutes.

### Exclusions

All components that are positively set forth in the instant disclosure may be negatively excluded from the claims, e.g., a claimed composition may be “free,” “essentially free” (or “substantially free”) of one or more components that are positively set forth in the instant disclosure.

In various embodiments, the hair straightening composition is free or essentially free from anionic surfactants.

In various embodiments, the hair straightening composition is free or essentially free from polymers, copolymers, and crosspolymers formed with acrylate or methacrylate monomers, e.g, free or essentially free from polyacrylic acid and polyacrylate polymers and crosspolymers.

In various embodiments, the hair straightening composition is free or essentially free from N-alkyl-2-mercaptoacetamide. In further embodiments, the hair straightening composition is free or essentially free from all mercaptoacetamides.

In various embodiments, the hair straightening composition is free or essentially free from ethylene carbonate. In further embodiments, the hair straightening composition is free or essentially free from free from linear carbonates, e.g., dimethyl carbonate, diethyl carbonate, etc. In various embodiments, the composition is free or essentially free from cyclic lactones (e.g., valerolactone, caprolactone, pantolactone, meadowlactone, etc.), free or essentially free from heterocyclic molecules (e.g., 2-oxazolidinone, 2-imidazolidinone, etc.) free or essentially free from sulfones (dimethyl sulfone, 2,3,4,5-tetrahydrothiophene-1,1-dioxide), and/or free or essentially free from ureas (e.g., urea, ethylene urea, etc.). In yet further embodiments, the hair straightening composition is free or essentially free from carbonates. For example, the hair straightening composition may be free or essentially free from cyclic carbonates, preferably cyclic carbonates selected from propylene carbonate, dipropylene carbonate, butylene carbonate, 2,3-butylene carbonate, 2,3-pentylene carbonate, pentylene carbonate, ethylene carbonate, glycerol carbonate, or a mixture thereof.

In various embodiments, the hair straightening compositions are free or essentially free from polysaccharides other than the cyclodextrin and/or derivatives thereof and the cationic guar derivatives.

In various embodiments, the hair straightening composition is free or essentially free from silicones, including amino-functionalized silicones such as amodimethicone.

In further embodiments, the hair straightening composition is free or essentially free from monosaccharides and disaccharides. For example, the composition is free or essentially free from ribose, arabinose, glucose, fructose, xylose, sucrose, and/or methyl glucoside.

In various embodiments, the hair straightening composition is free or essentially free from formaldehyde, derivatives of formaldehyde, formalin, and compounds that produce formaldehyde upon heating.

In further embodiments, the hair straightening composition is free or essentially free from thioglycolic acid, thiolactic acid, or salts thereof.

### EMBODIMENTS

In various embodiments, the hair straightening composition comprises or consists of:
(a) about 1 to about 8 wt. %, preferably about 1 to about 6 wt. %, more preferably about 2 to about 5 wt. % of citric acid, salt thereof, or combination thereof;
(b) about 0.5 to about 5 wt. %, preferably about 1 to about 5 wt. %, more preferably about 1 to about 3 wt. % of one or more cyclodextrins, derivatives thereof, or a combination thereof;
   wherein a combined total amount of the citric acid, salt thereof, or combination thereof of (a) and the one or more cyclodextrins, derivatives thereof, or combination thereof of (b) is about 2 to about 12 wt. %, preferably about 2 to about 8, more preferably about 3 to about 6 wt. %; and
   wherein preferably (a) and (b) are individually combined with each other to form a composition in which (b) is dissolved in (a) before subsequently being combined with additional components of the hair-treatment composition;
(c) about 0.1 to about 5 wt. %, preferably about 0.1 to about 3 wt. %, more preferably about 0.5 to about 2 wt. % of one or more water soluble solvents, wherein preferably the one or more water soluble solvents are selected from C₂-C₆ mono-alcohols (e.g., ethanol, isopropyl alcohol, etc.), polyols (polyhydric alcohols, such as glycerin), glycols (e.g., ethylene glycol, propylene glycol, butylene glycol, pentylene glycol, caprylyl glycol, etc.), and mixtures thereof, wherein more preferably at least one of the one or more water soluble solvents is glycerin;
(d) about 0.1 to about 8 wt. %, preferably about 0.2 to about 5 wt. %, more preferably about 0.5 to about 4 wt. % of one or more cationic guar derivatives, wherein the one or more cationic guar derivatives are preferably selected from cationic hydroxyethyl guar, cationic hydroxypropyl guar, cationic hydroxybutyl guar, cationic carboxymethyl guar, cationic carboxylpropyl guar, cationic carboxybutyl guar, carboxymethylhydroxypropyl guar, guar hydroxypropyltrimonium chloride, and hydroxypropyl guar hydroxypropyltrimonium chloride, preferably wherein the one or more cationic guar derivatives is guar hydroxypropyltrimonium chloride, hydroxypropyl guar hydroxypropyltrimonium chloride, or a mixture thereof;
(e) about 70 to about 96 wt. %, preferably about 80 to about 95 wt. %, more preferably about 85 to about 95 wt. % of water;
   wherein all percentages by weight are based on a total weight of the composition;
(f) optionally, about 0.1 to about 15 wt. %, preferably about 0.5 to about 10 wt. %, more preferably about 0.5 to about 5 wt. % of one or more water soluble solvents, preferably wherein the one or more water soluble solvents are selected from C₂-C₆ mono-alcohols, polyols (polyhydric alcohols), glycols, or a mixture thereof; and
(g) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof;
   wherein all percentages by weight are based on a total weight of the composition.

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, about 3 to about 4.5, about 3.5 to about 6.5, about 3.5 to about 6, about 3.5 to about 5.5, about 3.5 to about 5, or about 3.5 to about 4.5.

In various embodiments, the hair straightening composition comprises, consists essentially of, or consists of:
(a) about 1 to about 8 wt. %, preferably about 1 to about 6 wt. %, more preferably about 2 to about 5 wt. % of citric acid, salt thereof, or combination thereof;
(b) about 0.5 to about 5 wt. %, preferably about 1 to about 5 wt. %, more preferably about 1 to about 3 wt. % of one or more cyclodextrins, derivatives thereof, or a combination thereof;
   wherein a combined total amount of the citric acid, salt thereof, or combination thereof of (a) and the one or more cyclodextrins, derivatives thereof, or combination thereof of (b) is about 2 to about 12 wt. %, preferably about 2 to about 8, more preferably about 3 to about 6 wt. %;
   wherein preferably (a) and (b) are individually combined with each other to form a composition in which (b) is dissolved in (a) before subsequently being combined with additional components of the hair-treatment composition;
(c) about 0.1 to about 5 wt. %, preferably about 0.1 to about 3 wt. %, more preferably about 0.5 to about 2 wt. % of one or more water soluble solvents, wherein preferably the one or more water soluble solvents are selected from C₂-C₆ mono-alcohols (e.g., ethanol, isopropyl alcohol, etc.), polyols (polyhydric alcohols, such as glycerin), glycols (e.g., ethylene glycol, propylene glycol, butylene glycol, pentylene glycol, caprylyl glycol, etc.), and mixtures thereof, wherein more preferably at least one of the one or more water soluble solvents is glycerin;
(d) about 0.1 to about 8 wt. %, preferably about 0.2 to about 5 wt. %, more preferably about 0.5 to about 4 wt. % of guar hydroxypropyltrimonium chloride, hydroxypropyl guar hydroxypropyltrimonium chloride, or a mixture thereof;
(e) about 70 to about 96 wt. %, preferably about 80 to about 95 wt. %, more preferably about 85 to about 95 wt. % of water;
   wherein all percentages by weight are based on a total weight of the composition;
(f) optionally, about 0.1 to about 15 wt. %, preferably about 0.5 to about 10 wt. %, more preferably about 0.5 to about 5 wt. % of one or more water soluble solvents, preferably wherein the one or more water soluble solvents are selected from C₂-C₆ mono-alcohols, polyols (polyhydric alcohols), glycols, or a mixture thereof; and
(g) optionally, about 0.01 to about 10 wt. %, preferably about 0.1 to about 8 wt. %, more preferably about 1 to about 5 wt. % of one or more miscellaneous ingredients, preferably selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof;
   wherein all percentages by weight are based on a total weight of the composition.

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, about 3 to about 4.5, about 3 to about 4, about 3.5 to about 6.5, about 3.5 to about 6, about 3.5 to about 5.5, about 3.5 to about 5, or about 3.5 to about 4.5.

Implementation of the present disclosure is provided by way of the following examples. The examples serve to illustrate the technology without being limiting in nature.

### Example 1

### Compositions

| | | Inventive | Comparative | | | |
|---|---|---|---|---|---|---|
| | | A | B | C | D | E |
| (a) | Citric Acid | 2.5 | 2.5 | 2.5 | 2.5 | 2.5 |
| (b) | Cyclodextrin | 1.5 | 1.5 | 1.5 | 1.5 | 1.5 |
| Total (a) + (b) | | 4 | 4 | 4 | 4 | 4 |
| Weight Ratio | | 1.7:1 | 1.7:1 | 1.7:1 | 1.7:1 | 1.7:1 |
| Mole Ratio | | 10:1 | 10:1 | 10:1 | 10:1 | 10:1 |
| (c) | Glycerin | 1 | | 1 | 1 | 1 |
| (d) | Hydroxypropyl Guar | 1 | 1 | | | |
| | Hydroxy- | | | | | |
| | propyltirmonium | | | | | |
| | Chloride | | | | | |
| | Guar Gum | | | | 1 | |
| | Carrageenan | | | | | 1 |
| | Miscellaneous * | ≤5 | ≤5 | ≤5 | ≤5 | ≤5 |
| (e) | Water | 94 | 95 | 95 | 94 | 94 |

| | | | | | |
|---|---|---|---|---|---|
| pH | 3-4 | 3-4 | 3-4 | 3-4 | 3-4 |

* Fillers, pH adjusters, preservatives, botanical extract, fragrances, buffers, stabilizers, vitamins, and/or emollients, etc., for example, henoxyethanol, sodium hydroxide, and acetic acid.

### Example 2

### Straightening Performance without Heat

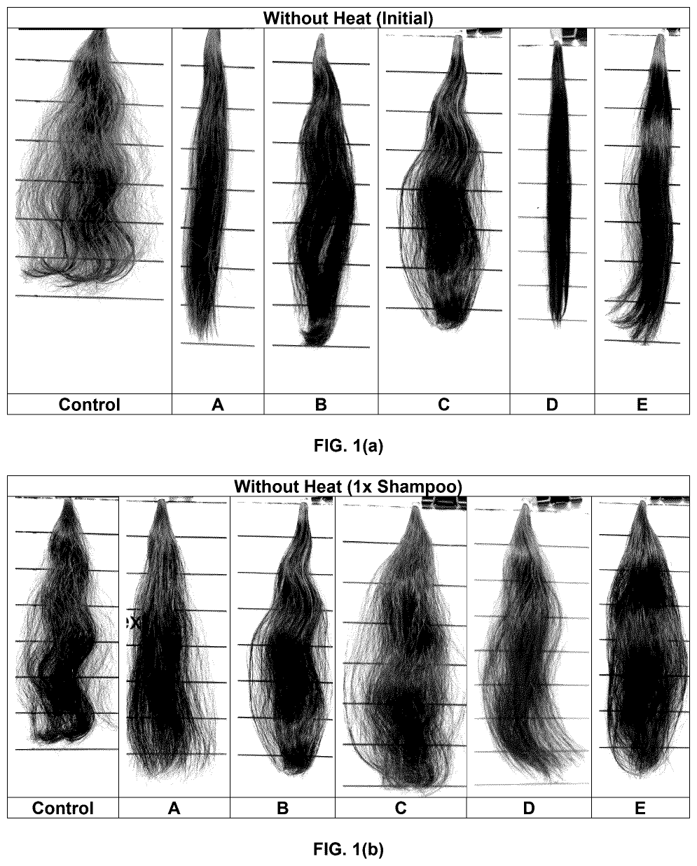

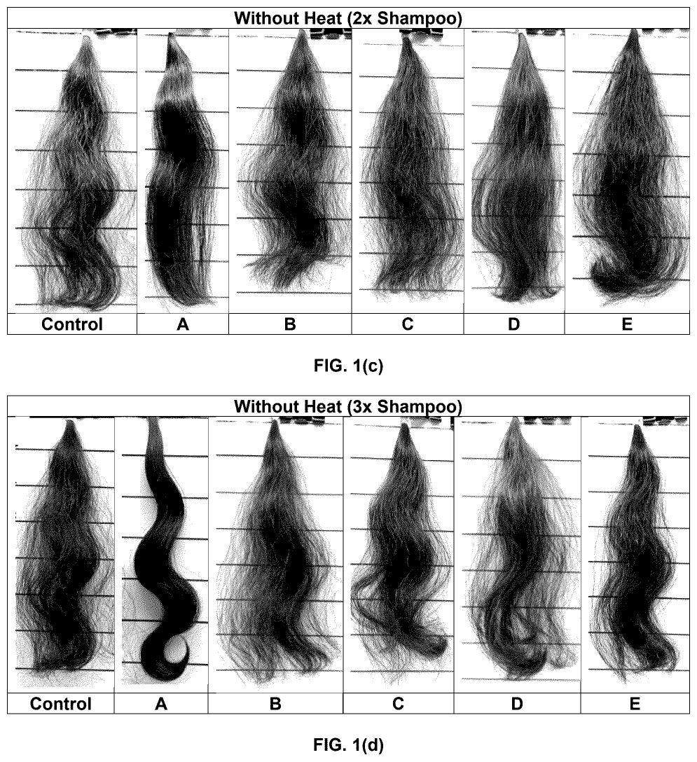

Inventive composition A and comparative compositions B-E were tested for their ability to straighten hair. Caucasian hair swatches (curl pattern 4) were obtained and cleansed with a standard shampoo. After rinsing the shampoo from the hair swatches, 0.4 grams (per gram of hair) of inventive composition A or comparative compositions B-E were applied to a hair swatch while wet. The hair swatches were combed six times to straighten and align the hair fibers, and to spread the various compositions throughout the hair fibers. For comparison, a control hair swatch was used that was cleansed with the same standard shampoo but not treated with inventive composition A or comparative compositions B-E. After rinsing the shampoo from the control hair swatch, while the hair was wet, the control hair swatch was combed six times to straighten and align the hair fibers. The hair swatches treated with inventive composition A, comparative compositions B-E, and the control were hung and allowed to dry naturally under ambient conditions (about 2 hours). The swatches were photographed and are shown in FIG. 1(a).

The hair swatches were then cleansed with the standard shampoo (1× wash), combed six times to straighten and align the hair fibers, hung, and allowed to dry naturally under ambient conditions (about 2 hours). The swatches were photographed and are shown in FIG. 1(b).

The hair swatches were again cleansed with the standard shampoo (2× wash), combed six times to straighten and align the hair fibers, hung, and allowed to dry naturally under ambient conditions (about 2 hours). The swatches were photographed and are shown in FIG. 1(c).

The hair swatches were further cleansed with the standard shampoo (3× wash), combed six times to straighten and align the hair fibers, hung, and allowed to dry naturally under ambient conditions (about 2 hours). The swatches were photographed and are shown in FIG. 1(d).

The results show that treatment with inventive composition A dramatically straightened the hair fibers without heat, and resulted in improved fiber alignment and frizz control (no frizz). These effects were retained even after the hair swatch was cleansed with a shampoo. As shown in FIG. 1(d), after being cleansed with a shampoo three times, the straightening effects dissipate but the hair fibers remained aligned and did not frizz. Hair swatches treated with inventive composition A were straighter, more aligned, and less frizzy than hair treated with the control and hair treated with the comparative compositions.

### Example 3

### Straightening Performance with Heat

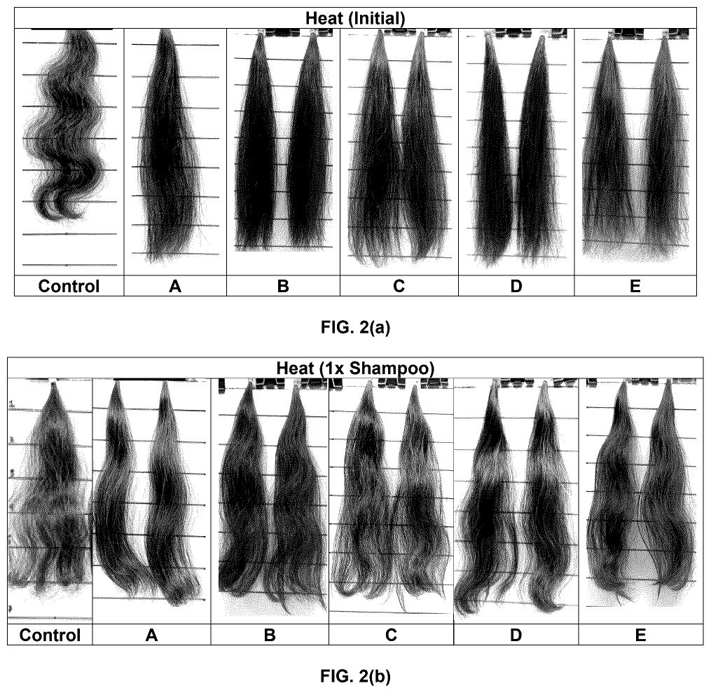

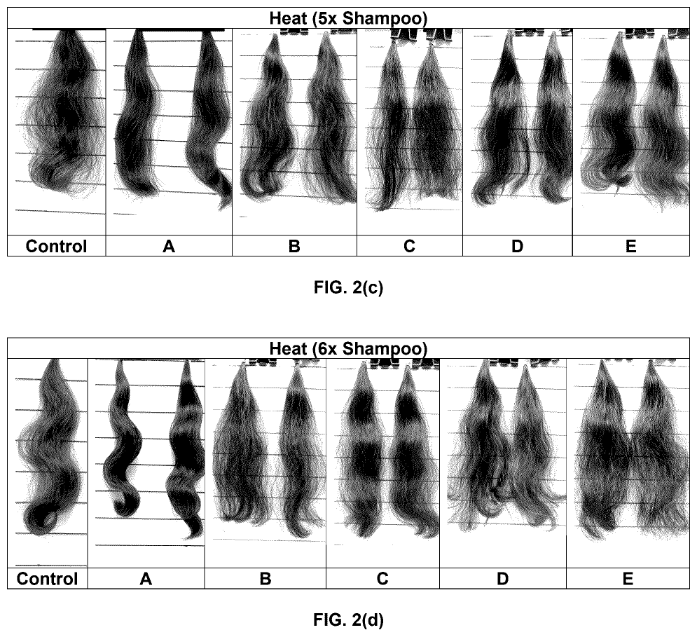

Inventive composition A and comparative compositions B-E were tested for their ability to straighten hair in combination with a heat treatment. Caucasian hair swatches (curl pattern 4) were obtained and cleansed with a standard shampoo. After rinsing the shampoo from the hair swatches, 0.4 grams (per gram of hair) of Inventive composition A or comparative composition B-E were applied to two hair swatches (n=2) while the hair was wet. The hair swatches were combed six times to straighten and align the hair fibers, and to spread inventive composition A throughout the hair fibers. For comparison, a control hair swatch was cleansed with the same standard shampoo. After rinsing the shampoo from the control hair swatch, while the hair was still wet, the control hair swatch was combed six times to straighten and align the hair fibers. The hair swatches were hung and allowed to dry naturally under ambient conditions for about 1 hour. Then, the hair swatches were straightened using a flat iron at a temperature of about 180° C. The flat iron was passed over the hair swatches 6 times (for about 10 seconds per pass). The swatches were photographed and shown in FIG. 2(a).

The hair swatches were subsequently cleansed with the standard shampoo (1× wash), combed six times to straighten and align the hair fibers, hung, and allowed to dry naturally under ambient conditions (about 2 hours). The swatches were photographed and are shown in FIG. 2(b).

The hair swatches were cleansed and dried repeatedly, in a cycle. After five cycles of cleansing and drying, the swatches were photographed and are shown in FIG. 2(c).

The cleansing and drying cycle was carried out a sixth time and pictures of the swatches are shown in FIG. 2(d).

The results show that a heat-treatment in combination with treating hair with inventive composition A enhances the hair straightening effects, with improved fiber alignment and frizz control (no frizz). These effects were retained even after the hair swatches were cleansed with a shampoo. As shown in FIG. 2(d), after being cleansed with a shampoo six times, the straightening effects dissipate but the hair fibers remained aligned and did not frizz. Hair swatches treated with inventive composition A were straighter, more aligned, and less frizzy than hair treated with the control and hair treated with the comparative compositions.

### Example 4

### Straightening Performance

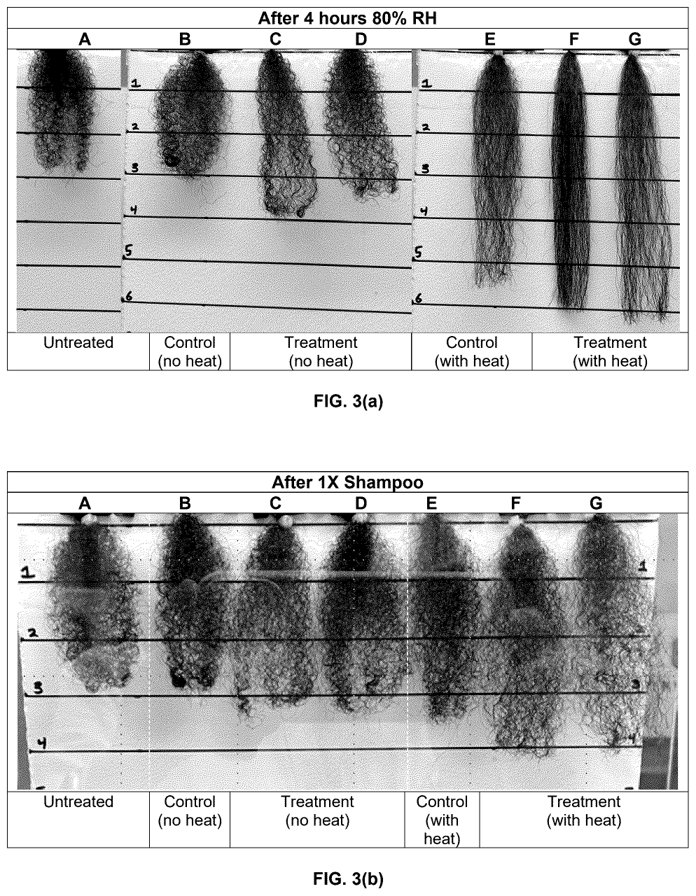

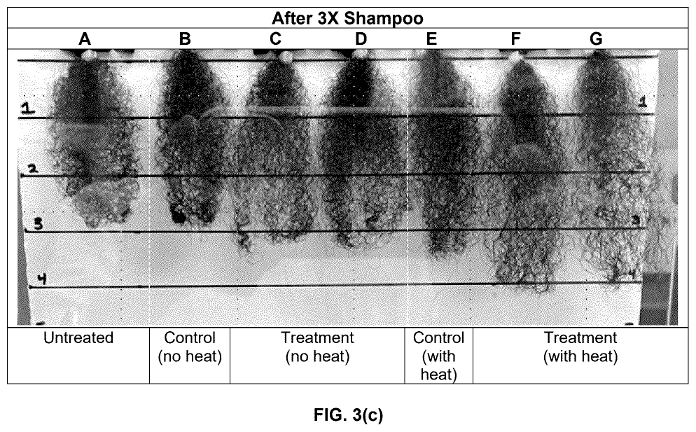

Testing was carried out on hair swatches (curl pattern 7) according to the straightening procedures described above in Example 3 (without heat) and Example 4 (with heat). After initial treatment, however, the hair swatches were placed in a humidity chamber for 4 hours at 80% relative humidity. Picture of the swatches after 4 hours in the humidity chamber appear in FIG. 3(a). Pictures of the hair swatches after 1 cycle of post-treatment washing and drying is shown in FIG. 3(b). Pictures of the hair swatches after 3 cycles of post-treatment washing and drying is shown in FIG. 3(c). The table below identifies how swatches A-G were treated.

| | |
|---|---|
| A | Untreated Hair Swatch |
| B | Control Swatch Without Heat |
| C & D | Treated Swatches Without Heat |
| E | Control Swatch With Heat |
| F & G | Treated Swatches With Heat |

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both a nonionic surfactant or emulsifier and a fatty compound. If a particular composition includes both a nonionic surfactant or emulsifier and a fatty compound, a single compound will serve as only the nonionic surfactant or emulsifier or only as the fatty compound (the single compound does not simultaneously serve as both the nonionic surfactant or emulsifier and the fatty component).

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The composition of the instant case optionally include one or more surfactants and/or emulsifiers, for example, one or more nonionic, anionic, cationic, and/or amphoteric/zwitterionic surfactants. The term “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.”

Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair straightening composition comprising:
(a) about 1 to about 8 wt. % of citric acid, a salt thereof, or a combination thereof;
(b) about 0.5 to about 5 wt. % of one or more cyclodextrins, derivatives thereof, or a combination thereof;
wherein a combined total amount of (a) and (b) is about 2 to about 12 wt. %;
(c) about 0.1 to about 5 wt. % of one or more water soluble solvents;
(d) about 0.1 to about 8 wt. % of one or more cationic guar derivatives;
(e) about 70 to about 96 wt. % of water;
wherein all percentages by weight are based on a total weight of the composition.

2. The composition of claim 1 having a pH of about 3 to about 6.

3. The composition of claim 1, wherein (a) and (b) are in a weight ratio of about 4:1 to about 1:1 ((a):(b)) and/or a molar ratio of about 20:1 to about 5:1 ((a):(b)).

4. The composition of claim 1, wherein (a) and (b) are individually combined with each other to form a mixture in which (b) is dissolved (a); and the mixture is subsequently combined with additional components of the composition.

5. The composition of claim 1, wherein the one or more cyclodextrins, derivatives thereof, or a mixture thereof, are selected from α-cyclodextrin, β-cyclodextrin, γ-cyclodextrin, methyl-α-cyclodextrin, methyl-β-cyclodextrin, methyl-γ-cyclodextrin, or mixtures thereof.

6. The composition of claim 1, wherein the one or more cyclodextrins, derivatives thereof, or a mixture thereof are selected from β-cyclodextrin, derivatives thereof, or mixtures thereof.

7. The composition of claim 1, wherein the composition is free from film-forming polymers.

8. The composition of claim 1, further comprising:
(f) about 0.1 to about 10 wt. % of one or more miscellaneous ingredients.

9. The composition of claim 8, wherein the one or more miscellaneous ingredients are selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or mixtures thereof.

10. A hair straightening composition comprising:
(a) about 1 to about 5 wt. % of citric acid, salt thereof, or combination thereof;
(b) about 1 to about 5 wt. % of one or more cyclodextrins, derivatives thereof, or a combination thereof;
wherein a combined total amount of (a) and (b) is about 2 to about 8 wt. %;
(c) about 0.5 to about 4 wt. % of one or more water soluble solvents;
(d) about 0.5 to about 5 wt. % of hydroxypropyl guar hydroxypropyltirmonium chloride;
(e) about 70 to about 96 wt. % of water;
(f) optionally, about 0.1 to about 10 wt. % of one or more miscellaneous ingredients;
wherein the composition has a pH of about 3 to about 5, and all percentages by weight are based on a total weight of the composition.

11. The composition of claim 10, wherein (a) and (b) are in a weight ratio of about 4:1 to about 1:1 ((a):(b)) and/or a molar ratio of about 20:1 to about 5:1.

12. The composition of claim 11, wherein (a) and (b) are individually combined with each other to form a mixture in which the cyclodextrin is dissolved in the citric acid; and the mixture is subsequently combined with additional components of the composition.

13. The composition of claim 11, wherein the one or more cyclodextrins, derivatives thereof, or a mixture thereof, are selected from β-cyclodextrin, derivatives thereof, or mixtures thereof.

14. The composition of claim 11, wherein the one or more miscellaneous ingredients are selected from preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or mixtures thereof.

15. A method for straightening hair comprising:
(i) applying the hair straightening composition of claim 1 to the hair;
(ii) physically manipulating the hair upon which the hair straightening composition is applied with a straightening action; and
(iii) without removing the straightening composition from the hair, drying the hair.

16. The method of claim 14, wherein physically manipulating the hair comprises combing or brushing the hair.

17. The method of claim 14, further comprising:
(iv) treating the hair with a flat iron at a temperature of about 140° C. to about 220° C., wherein the treatment with the flat iron is optionally preceded by a pause time of from 1 minute to about 1 hour.

18. The method of claim 14, wherein drying the hair comprises allowing the hair to dry naturally under ambient conditions or drying the hair with a blow dryer.

19. A method for making the hair straightening composition of claim 1 comprising:
(i) dissolving (b) in (a) to obtain a mixture; and
(ii) combining the mixture with additional component of the composition.

20. The method of claim 19, wherein (a) and (b) are in a weight ratio of about 4:1 to about 1:1 ((a):(b)) and/or a molar ratio of about 20:1 to about 5:1 ((a):(b)) and (a) and (b) are heated to a temperature of about 70 to about 120° C. for about 1 to about 5 hours to dissolve (b) in (a).
